(19) United States
(12) Patent Application Publication
(10) Pub. No.: US 2015/0004632 A1
(43) Pub. Date: Jan. 1, 2015

(54) DIAGNOSTIC AND PROGNOSTIC USE OF PROMBP-COMPLEXES

(71) Applicant: Aarhus Universitet, Aarhus C (DK)

(72) Inventors: Claus Oxvig, Viby J (DK); Soren Kloverpris, Risskov (DK)

(73) Assignee: AARHUS UNIVERSITET, Aarhus C, (DK)

(21) Appl. No.: 14/351,662

(22) PCT Filed: Oct. 12, 2012
(86) PCT No.: PCT/DK2012/000112

§ 371 Date: Apr. 14, 2014

(30) Foreign Application Priority Data
Oct. 14, 2011 (DK) PA 2011 70572

(51) Int. Cl. G01N 33/68

(52) U.S. Cl. CPC G01N 33/689; G01N 2800/368; G01N 2333/471
USPC 435/7.92

(57) ABSTRACT

The present Invention relates to a diagnostic and/or prognostic method comprising measurements of proMBP-complexes (major basic protein, PRG2). In particular the Invention relates to a diagnostic and/or prognostic method comprising measurements of the concentration of two or more proMBP-complexes. Further, the Invention relates to a method for determining the redox state In a pregnant female. Additionally, the Invention relates to a reaction kit comprising detection means for detecting two or more proMBP complexes, as well as to various uses of the reaction kit. Further, the invention relates to a method of diagnosis of preeclampsia by determining the ratio between proMBP protein in complex with PAPP-A and proMBP protein in complex with angiotensinogen (ATG).

### TECHNICAL FIELD OF THE INVENTION

The present invention relates to a diagnostic and/or prognostic method comprising measurements of proMBP-complexes. In particular the invention relates to diagnostic and/or prognostic methods comprising measurements of the concentration of two or more protein complexes between proMBP and other proteins.

### BACKGROUND OF THE INVENTION

Angiotensinogen (AGT) is a non-inhibitory serpin of approximately 60 kDa, known as the precursor of angiotensin-1, generated by renin cleavage. In addition to systemic effects of the renin-angiotensin system, local and tissue-specific effects, e.g. in the uteroplacental unit, have been described. The plasma concentration of AGT in non-pregnant women is close to the Km (1 μM) of its reaction with renin, but during pregnancy, it is increased up to four fold. In men and non-pregnant women, the majority of circulating AGT is monomeric. However, 3-5% is present in a poorly characterized high-molecular weight (HMW) form. In normotensive pregnant women, the HMW fraction is increased to approximately 16%, and under pathological conditions, such as pregnancy-induced hypertension and preeclampsia, it may become the predominant form.

In vitro, the AGT molecule is known to be able to form both non-covalent and disulphide linked multimers, thought to be the dominant constituent of the HMW fraction. In addition, the presence of two different complexes containing the proform of eosinophil major basic protein (proMBP) disulphide linked to AGT has been demonstrated. One is a 2:2 proMBP/AGT complex of approximately 200 kDa. The other is a ternary complex with complement C3dg, a 2:2:2 proMBP/AGT/C3dg complex, of approximately 300 kDa.

ProMBP is synthesized in large quantity by extravillous trophoblast in the placenta, and its concentration increases throughout pregnancy. It is known to circulate in a 2:2 disulphide-linked complex with the metzincin metalloproteinase, pregnancy-associated plasma protein-A (PAPP-A).

Thus, a network of complexes exists in which proMBP, AGT and PAPP-A are key elements. Although the proMBP/PAPP-A complex is well characterized, the situation regarding the important HMW AGT is until now unknown. Since both HMW AGT and proMBP/PAPP-A are known to play crucial roles in both normal and complicated pregnancies, there is a need for better and more precise diagnostic and prognostic tools that are able to determine diseases or complications, and being able to assess the disease state in the pregnant woman.

### SUMMARY OF THE INVENTION

Thus, an object of the present invention is to providing a valuable diagnostic and/or prognostic tool for determining disease, disease state and/or disease development in an individual.

In a first aspect, the present invention relates to a diagnostic and/or prognostic method comprising measurements of the concentrations of two or more different proMBP-complexes in an isolated sample from an individual.

In a second aspect, the present invention relates to a reagent kit comprising detection means for measuring the concentration and/or ratio of two or more different proMBP-complexes in an isolated sample from an individual.

Moreover, in a third aspect, the present invention relates to a method for determination of the redox state of a pregnant female comprising measurement of the concentration of and/or ratio between two or more proMBP-complexes.

In a fourth aspect, the present invention relates to the use of a reagent kit for assessing and/or monitoring the redox state in an individual.

Further, in a fifth aspect, the present invention relates to the use of a reagent kit as for diagnostic and/or prognostic purposes.

Lastly, in a sixth aspect, the present invention pertains to the use of a reagent kit for assessing and/or monitoring the disease state and/or disease development in an individual.

### BRIEF DESCRIPTION OF THE FIGURES

FIG. 1 shows the network of protein complexes present in the circulation of pregnant women.

FIG. 2 shows that proMBP is a major constituent of the high-molecular weight fraction of angiotensinogen (AGT) in late pregnancy plasma.

FIG. 3 shows that all proMBP is disulfide linked to PAPP-A or AGT in late pregnancy plasma.

FIG. 4 shows that complement C3dg containing complexes form as a consequence of post-sampling events in late pregnancy plasma and serum.

FIG. 5 shows that formation of the proMBP/PAPP-A and proMBP/AGT complexes are competing reactions whose balance/ratio depends on the redox potential.

FIG. 6: Serum concentrations of proMBP complexes before week 25 of gestation. A, concentrations of PAPP-A/proMBP and AGT/proMBP complexes were measured in pregnancy serum collected from women who developed preeclampsia (n=31) and a control group of normotensive pregnant women (n=161). Furthermore, (AGT/proMBP)/(PAPP-A/proMBP) was calculated for each serum sample. Box and whiskers plots are shown for all groups. The whiskers indicate the maximum and minimum, the line is the median, and the box represents the lower and the upper quartile. Groups were compared using the Mann-Whitney test (GraphPad Prism 5.00): * P<0.05; ** P<0.01; *** P<0.001. B, To evaluate the diagnostic value of the measured concentrations, ROC curves are shown. The true positive rate (Sensitivity) is plotted as a function of the false positive rate (1-Specificity), and the areas (A) under the curves was calculated and shown in the graph (GraphPad Prism 5.00).

FIG. 7: Serum concentrations of proMBP complexes from week 26 to week 29 of gestation.

A, concentrations of PAPP-A/proMBP and AGT/proMBP complexes were measured in pregnancy serum collected from women who developed preeclampsia (n=28) and a control group of normotensive pregnant women (n=119). Furthermore, (AGT/proMBP)/(PAPP-A/proMBP) was calculated for each serum sample. Box and whiskers plots are shown for all groups. The whiskers indicate the maximum and minimum, the line is the median, and the box represents the lower and the upper quartile. Groups were compared using the Mann-Whitney test (GraphPad Prism 5.00): * P<0.05; ** P<0.01; *** P<0.001. B, To evaluate the diagnostic value of the measured concentrations, ROC curves are shown. The true positive rate (Sensitivity) is plotted as a function of the false positive rate (1-Specificity), and the areas (A) under the curves was calculated and shown in the graph (GraphPad Prism 5.00).

FIG. 8: Serum concentrations of proMBP complexes from week 30 to week 35 of gestation.

A, concentrations of PAPP-A/proMBP and AGT/proMBP complexes were measured in pregnancy serum collected from women who developed preeclampsia (n=32) and a control group of normotensive pregnant women (n=149). Furthermore, (AGT/proMBP)/(PAPP-A/proMBP) was calculated for each serum sample. Box and whiskers plots are shown for all groups. The whiskers indicate the maximum and minimum, the line is the median, and the box represents the lower and the upper quartile. Groups were compared using the Mann-Whitney test (GraphPad Prism 5.00): * P<0.05; ** P<0.01; *** P<0.001. B, To evaluate the diagnostic value of the measured concentrations, ROC curves are shown. The true positive rate (Sensitivity) is plotted as a function of the false positive rate (1-Specificity), and the areas (A) under the curves was calculated and shown in the graph (GraphPad Prism 5.00).

### DEFINITIONS

“Preeclampsia”: Preeclampsia is a disorder in pregnant women of widespread vascular endothelial malfunction and vasospasm that usually occurs after 20 weeks gestation and can present as late as 4-6 weeks postpartum. It is clinically defined by hypertension and proteinuria, with or without pathologic edema. Medical consensus is lacking regarding the values that define preeclampsia; however, as used herein, “preeclampsia” is present in a woman who was normotensive before 20 weeks' gestation but develops a systolic blood pressure (SBP) greater than 140 mm Hg and/or a diastolic BP (DBP) greater than 90 mm Hg on 2 successive measurements, taken 4-6 hours apart. In a patient with preexisting essential hypertension (i.e., essential hypertension present before the pregnancy), preeclampsia is present if SBP has increased by 30 mm Hg or if DBP has increased by 15 mm Hg.

“Individual” means a pregnant female, preferably a pregnant female human being.

“Determining” the level of proMBP complexes means detection or measurement of the level of the proMBP protein complexes. Any suitable detection method or measurement can be used. The measurement can be qualitative, semi-quantitative and/or quantitative.

“Normal level of proMPB complexes” means a level of proMBP complexes in a population of pregnant women without a pregnant associated disease or condition such as without preeclampsia or without an identified risk of developing said disease of condition. The normal level of the proMBP complexes depends on the gestation time. Accordingly, the normal level has to be determined for each respective gestation time period, such as each week or range of weeks during the pregnancy.

“Threshold level” means a selected or identified level of one or more proMBP complexes that represents a particular status with respect to the diagnosis or prognosis at a respective gestation time period. If the level of one or more proMBP complexes is higher or lower than the threshold level it can indicate that the individual has a pregnant associated disease such as preeclampsia or is prognosed as having an increased risk of developing a pregnant associated disease such as preeclampsia.

In one embodiment the threshold level can be defined—for a certain gestation week—by multiplying or dividing the normal level of the one or more proMBP complexes in a certain gestation week with a “margin factor (x). In one embodiment the margin factor can be 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more than 10 or any interval limited by any two of these values.

“Secondary marker” is any marker other than proMBP-complexes that can be used to determine if a pregnant woman has a pregnancy associated disease or complication such as preeclampsia or an increased risk of developing said disease or complication such as preeclampsia. The secondary marker can be any marker disclosed in the prior art or disclosed in the present application. The combination of the diagnostic or prognostic method based on measurement of proMBP-complexes with measurements of the level of one or more secondary markers may increase the reliability of predicting the likelihood of having or developing a pregnancy associated disease or complication such as preeclampsia compared to using the measurements of the proMBP-complexes alone, or it may suggest additional treatment options, such as by identifying underlying or secondary mechanisms affecting the subject.

“More likely” in connection with use of one or more secondary marker(s) for diagnosing or prognosing a pregnancy associated disease or complication such as preeclampsia means that if both proMBP-complexes and the secondary marker give positive results, the diagnosis and/or prognosis of a pregnancy associated disease or complication such as preeclampsia is more reliable or more likely to be correct. This can mean in one embodiment that the number of false positives is reduced by e.g. more than 5%, more than 10%, more than 20%, more than 30%, more than 40%, more than 50%, more than 60%, more than 70%, more than 80 or even more than 90% compared to a method in which either the proMBP-complexes or the one or more secondary marker were measured. This can mean in another embodiment that the number of false negatives is reduced by e.g. more than 5%, more than 10%, more than 20%, more than 30%, more than 40%, more than 50%, more than 60%, more than 70%, more than 80 or even more than 90% compared to a method in which the proMBP-complexes or the one or more secondary marker(s) were used as the only marker(s).

“Diagnosed” as having a pregnancy associated disease or complication such as preeclampsia means that it has been determined, with some probability or margin of error, which may be defined or unknown, that the subject has a pregnancy associated disease or complication such as preeclampsia.

“Prognosed” as likely to develop preeclampsia means that it has been determined, with some probability or margin of error, which may be defined or unknown, that the subject has a predisposition to have or will have preeclampsia at a later gestation time during the pregnancy, although the subject most likely does not have preeclampsia at the time the prognosis is made.

### DETAILED DESCRIPTION OF THE INVENTION

The plasma concentration of placentally derived proform of eosinophil major basic protein (proMBP) increases throughout pregnancy, and three different complexes containing proMBP have been isolated from pregnancy plasma and serum: A 2:2 complex with the metalloproteinase, pregnancy-associated plasma protein-A (PAPP-A), a 2:2 complex with angiotensinogen (AGT), and a 2:2:2 complex with AGT and complement C3dg. PAPP-A is proteolytically inactive when complexed to proMBP, and renin cleavage of AGT in complex with proMBP progresses slower compared to cleavage of monomeric AGT. Within the context of the present invention the term “complex” refers to the covalently or non-covalently assembly of two or more molecules into one complex. In an embodiment of the present invention, the complexes are formed by covalent assembly, such as inter-molecular disulphide bonds.

The inventors of the present invention have through meticulous experimentation surprisingly established that all the proMBP circulating during human pregnancy is bound to either PAPP-A or AGT. The inventors have also established that the proMBP/AGT complex constitutes the major fraction of circulating high-molecular weight AGT in late pregnancy, and that this complex is able to associate with complement C3 derivatives post-sampling, forming a proMBP/AGT/C3dg complex. Furthermore, the inventors of the present invention have established that the proMBP/PAPP-A and the proMBP/AGT complexes are competing reactions, and that they depend differentially on the redox potential, which thus determines the relative amounts of the complexes.

The inventors of the present invention have therefore surprisingly found, that measuring the concentration ratios of proMBP complexes, and particularly of the proMBP/PAPP-A, proMBP/AGT and proMBP/AGT/C3dg complexes may serve as a very useful diagnostic and/or prognostic tool, since these ratios reflect the redox state in the pregnant woman, which the inventors of the present invention know is a diagnostic and/or prognostic indicator for pregnancy associated diseases and complications.

Thus, in one embodiment the present invention relates to a diagnostic and/or prognostic method comprising measurements of the concentrations of two or more different proMBP-complexes in an isolated sample from an individual.

However, in certain embodiments the present invention relates to a diagnostic and/or prognostic method comprising measurement of the concentration of one or more proMBP-complexes, such as measurement of the concentration of the proMBP/AGT complex and/or the proMBP/PAPP-A complex and/or the proMBP/AGT/C3dg complex.

These concentration measurements may be performed by any method known to the skilled person such as, but not limited to, quantitative immunochemistry assays, e.g. Enzyme-linked immunosorbent assay (ELISA) or quantitative western immunoblotting, Bradford and Bradford ULTRA assays, bicinchoninic acid (BCA) assay and quantitative mass spectrometry, such as quantitative Q-TOF mass spectrometry or quantitative MALDI-TOF mass spectrometry, or measurement of specific PAPP-A activity, since uncomplexed PAPP-A is active, while PAPP-A complexed to proMBP is completely inactive. In one embodiment of the present invention, the concentration measurements are performed using the Enzyme-linked immunosorbent assay (ELISA). In another embodiment, the concentration measurements are performed by measuring the PAPP-A activity. In a certain embodiment of the present invention, the concentration measurements are a combination of ELISA measurements and measurements of the PAPP-A activity.

In the present context the term “concentration” refers to any expression of concentration known to the person skilled in the art, such as weight concentration (g/l) molar concentration (mol/l=M), molal concentration (mol/kg), International Unit (IU) concentration, activity (g/I or mol/l), weight percentage and mol percentage.

The concentration of the proMBP-complexes according the invention will to some extend vary, not only form individual to individual, but also during development of the pregnancy. Hence, the concentration of the proMBP-complexes according to the present invention is in the range of 0-5000 nM, such as 0-4000 nm, e.g. 0-3000 nM, such as 0-2000 nM, for example 0-1000 nM, such as 0-500 nM.

It is within the scope of the invention, that all the concentration and/or ratio measurements described herein may not only be indicative of disease, disease state or disease development, but all the measurements may also be predictive, i.e. being able to predict future disease development in the individual. In this aspect, the present invention serves as a prognostic tool.

As described above the inventors of the present invention have surprisingly established that proMBP always is bound in complex with one or more other proteins, and that these one or more other proteins are PAPP-A, AGT and complement C3dg. Hence, in one embodiment the present invention relates to a diagnostic and/or prognostic method comprising measurements of the concentrations of proMBP-complexes, wherein the complexes are proMBP/AGT and proMBP/PAPP-A.

In another embodiment the present invention relates to a diagnostic and/or prognostic method comprising measurements of the concentrations of proMBP-complexes, wherein the complexes are proMBP/AGT and proMBP/AGT/C3dg.

In yet another embodiment the present invention relates to a diagnostic and/or prognostic method comprising measurements of the concentrations of proMBP-complexes, wherein the complexes are proMBP/PAPP-A and proMBP/AGT/C3dg.

In a further embodiment the present invention relates to a diagnostic and/or prognostic method comprising measurements of the concentrations of proMBP-complexes, wherein the complexes are proMBP/AGT and proMBP/PAPP-A and proMBP/AGT/C3dg.

Since the inventors of the present invention have established that the exact concentrations of two or more of the proMBP/PAPP-A, proMBP/AGT and proMBP/AGT/C3dg complexes are related to pregnancy associated diseases and conditions, the present invention also pertains to a diagnostic and/or prognostic method comprising measurements of two or more proMBP-complexes, wherein the concentrations of the individual complexes are indicative of a disease and/or a disease state and/or disease development. In a certain embodiment the present invention relates to a diagnostic and/or prognostic method comprising measurements of the proMBP/PAPP-A and proMBP/AGT complexes, wherein the concentrations of these individual complexes are indicative of a disease and/or a disease state and/or disease development.

As earlier described the proMBP protein is always complexed to one or more other proteins, and therefore measuring the concentration ratios between the individual complexes may be a valuable tool for determining the balance between the different complexes, which again may serve as a valuable diagnostic and/or prognostic tool. Thus the present invention relates to a diagnostic and/or prognostic method comprising measurements of two or more proMBP-complexes, wherein the ratio between the concentrations of the individual complexes is indicative of disease and/or disease state and/or disease development.

The term ratio refers in the present context to the concentration of one individual proMBP complex divided by the total concentration of a different proMBP-complexes. Since the inventors of the present invention surprisingly have established that all present proMBP is complexed, the total cumulative concentration of all the proMBP-complexes equals the total proMBP concentration.

The term balance refers in the present context to the concentration of one individual proMBP complex divided by cumulative concentration of all proMBP complexes.

In a one embodiment the present invention relates to a diagnostic and/or prognostic method comprising measurements of two or more proMBP-complexes, wherein a higher concentration of proMBP/AGT compared to the concentration of proMBP/PAPP-A is indicative of disease and/or disease state and/or disease development.

In another certain embodiment present invention relates to a diagnostic and/or prognostic method comprising measurements of two or more proMBP-complexes, wherein a higher concentration of proMBP/PAPP-A compared to the concentration of proMBP/AGT is indicative of disease and/or disease state and/or disease development.

In a certain embodiment present invention relates to a diagnostic and/or prognostic method comprising measurements of two or more proMBP-complexes wherein a change in ratio between the concentration of proMBP/PAPP-A and the concentration of proMBP/AGT is indicative of disease and/or disease state and/or disease development.

The concentration and/or ratio of the different proMBP-complexes is not static in an individual, and hence not static in a sample from an individual, and therefore the concentration and/or ratio of the proMBP-complexes may increase or decrease over time. Hence, the terms “change/shift in ratio” or “change/shift in concentration” or “change/shift in balance” refer in the present context to either a decrease or increase in ratio and/or concentration of the different proMBP-complexes, when comparing two or more consecutive measurements of the complexes over time, such as 3 consecutive measurements, for example 4 consecutive measurements, such as 5 consecutive measurements, e.g. 6 consecutive measurements, such as 7 consecutive measurements, for example 8 consecutive measurements, e.g. 9 consecutive measurements, such as 10 consecutive measurements, such as 15 consecutive measurements over time.

However, the terms “change/shift in ratio” or “change/shift in concentration” or “change/shift in balance” may in the present context also refer to either a decrease or increase in ratio and/or concentration of the different proMBP-complexes, when comparing to one or more starting values, such as one or more normal values.

In an embodiment, the present invention relates to a diagnostic and/or prognostic method comprising measurements of two or more proMBP-complexes, wherein a shift in ratio, such as a shift in ratio over time, between the two or more different proMBP-complexes is indicative of disease and/or a change in disease state and/or disease development.

Since the present invention relates to measurements of different compounds at different points in time, wherein the measurements may be compared to one or more earlier measurements, and/or one or more normal values, the present invention also relates to a diagnostic and/or prognostic multivariant analysis method, said method comprising one or more algorithms for calculating and predicting the disease development in and individual based on the multivariant analysis.

The disease development may either relate to a worsening, i.e. development towards a progressed disease state, or the disease development may relate to a bettering, i.e. development towards a relieved disease state. Therefore, the present invention pertains to a diagnostic and/or prognostic method comprising measurements of two or more proMBP-complexes, wherein the disease development pertains to a development towards a relieved disease state or towards a progressed disease state.

Complex formation between proMBP and AGT, resulting in proMBP/AGT and between proMBP and PAPP-A, resulting in proMBP/PAPP-A, depends on the redox potential. Therefore, determination of the extent of complex formation of individual complexes and/or the determination of ratios between formed complexes may reflect the average redox potential in the micro-environment of tissues where the covalent complexes are formed. The redox potential may be affected by changes in oxygen tension, e.g. hypoxia, often associated with pregnancy. Thus, in the present context the term “redox state” refers to a qualitative and/or quantitative measure of the average redox potential, or average redox potential over time, which may influence the formation of the proMBP-complexes.

Hence, the present invention pertains in one embodiment to a diagnostic and/or prognostic method comprising measurements of two or more proMBP-complexes, wherein the concentrations of the individual complexes are indicative for the redox state in an individual.

In another embodiment the present invention relates to a diagnostic and/or prognostic method comprising measurements of two or more proMBP-complexes, wherein the ratios between the two or more different proMBP-complexes are indicative for the redox state in an individual.

In yet another embodiment the present invention relates to a diagnostic and/or prognostic method comprising measurements of two or more proMBP-complexes, wherein a shift in ratio, such as shift in ratio over time, between the two or more different proMBP-complexes is indicative for a change the redox state in an individual.

Since the redox potential may be affected by changes in oxygen tension, the concentration and/or ratio of the individual proMBP-complexes may be subject to change under conditions wherein the oxygen tension is affected. Therefore, the present invention relates to a diagnostic and/or prognostic method comprising measurements of two or more proMBP-complexes, wherein the redox state or the change in redox state is caused by conditions affecting the oxygen tension, such as hypoxia, hyperoxia and hypertension.

The diagnostic and/or prognostic method of the present invention may be suitable for any disease and/or condition, wherein proMBP-complexes play a role. Additionally, diseases and/or conditions wherein other components that are able to interact with proMBP-complexes play a role, may also be suitable target diseases/conditions of the present invention. In an embodiment, the present invention pertains to a diagnostic and/or prognostic method comprising measurements of two or more proMBP-complexes, wherein the disease is a pregnancy associated disease and/or a pregnancy associated complication and/or a genetic abnormality.

Suitable target diseases and/or complications according to the present invention may be pregnancy associated diseases or complications selected from the group of diseases/complications consisting of small-for-gestational age, preterm delivery, IUGR (intrauterine growth restriction), miscarriage, stillbirth, gestational hypertension, HELLP (Haemolysis, Elevated Liver Enzymes and Low Platelet count), preeclampsia and any combination thereof.

Suitable target genetic abnormalities according to the invention may be genetic abnormality is selected from the group of genetic abnormalities consisting of trisomy 13, trisomy 18, trisomy 21 and any combination thereof.

It is within the scope of the present invention that the diagnostic and/or prognostic method comprising measurements of two or more proMBP-complexes may be performed on any object or entity. However, in an embodiment the measurements are performed in or on an isolated sample from a human being or an animal. In a preferred embodiment of the present invention, the measurements of the two or more proMBP-complexes are performed in or on an isolated sample from a pregnant female individual and/or a fetus. In a certain embodiment of the present invention, the measurements of two or more proMBP-complexes are simultaneously performed in or on isolated samples from a pregnant female individual and a fetus

The isolated sample according to the invention may be any sample, e.g. a biological fluid and/or a tissue biopsy, suitable for measuring the two or more proMBP-complexes. Suitable fluids pertains to any biological fluid, such as a blood sample, a serum sample, a plasma sample, a cerebrospinal fluid sample, and an amniotic fluid sample. Suitable tissue biopsies pertain to any tissue biopsy, such as a placental biopsy or a biopsy from the umbilical cord.

Thus, the present invention relates to a diagnostic and/or prognostic method comprising measurements of two or more proMBP-complexes, wherein the method comprises the steps of:
a. obtaining an isolated sample from an individual;
b. measuring the concentration and/or ratio of two or more different proMBP-complexes in said isolated sample;
c. obtaining the result; and
d. using the result of step c. to assess and/or determine the disease, the disease state and/or disease development of said individual.

As stated herein above, an object of the method according to the invention in determining the disease development in an individual, is be able to predict future disease development in the individual. In this sense, the method according to the invention is both a diagnostic method and a prognostic method.

As described elsewhere herein the redox state may affect the concentrations and/or ratios of the proMBP-complexes of the present invention. Accordingly, by measuring the concentrations and/or the ratios of the complexes, the inventors have been able to determine the redox state in an individual, such as in a pregnant woman.

Therefore, the present invention additionally relates to a method for determination of the redox state of a pregnant female comprising measurement of the concentration of and/or ratio between two or more proMBP-complexes.

In a preferred embodiment the present invention relates to a method for determination of the redox state in a pregnant woman by measuring the concentrations and/or the ratios of the proMBP/AGT complex and the proMBP/PAPP-A complex.

The present invention further relates to a reagent kit for measuring the concentration and/or ratio of two or more different proMBP-complexes in an isolated sample from an individual.

In an embodiment of the present invention, the reagent kit relates to a kit comprising detection means for performing an immunoassay. The reagent kit of the present invention may be a reagent kit for use in an immunoassay according to the Enzyme-linked Immunosorbent Assay (ELISA) method, wherein the reagent kit may comprise (i) one or more primary antibodies, (ii) one or more enzyme-labeled secondary antibodies, (iii) antibody-fixed beads, (iv) an assay buffer, (v) a substrate solution, (vi) a color forming agent, (vii) a color formation stopping solution, (viii) a standard, (ix) a washing solution, or any combination thereof. It is however within the scope of the present invention that the reagent kit may comprise fewer of the above components. In one embodiment, the reagent kit according to the invention comprise only (i) primary antibodies.

The reagent kit of the present invention is particular useful for performing complex-specific ELISAs, wherein the primary antibodies serve as detection means, such as antigen detection means. Hence, the primary antibodies of the reagent kit of the present invention are able bind either to the individual molecular components of the complexes, i.e. proMBP, or AGT, or PAPP-A, or complement C3dg in a complex-specific manner, or the primary antibodies are able to bind to the assembled complexes, i.e. proMBP/AGT, or proMBP/PAPP-A, or proMBP/AGT/C3dg.

Thus in one embodiment, the present invention relates to a reagent kit comprising detection means, wherein the detection means are antibodies directed towards proMBP and/or antibodies directed towards AGT and/or antibodies directed towards PAPP-A and/or antibodies directed towards complement C3dg.

In another embodiment, the present invention relates to a reagent kit comprising detection means, wherein the detection means are antibodies directed towards the two or more proMBP-complexes, i.e. antibodies directed towards the proMBP/AGT complex and/or antibodies directed towards the proMBP/PAPP-A complex and/or antibodies directed towards the proMBP/AGT/C3dg complex.

In the present context, the term “antibody” relates to any type of antibody known to the skilled person which is capable of recognizing and binding to an antigen. Suitable antibodies of the present invention include, but are not limited to, monoclonal antibodies, polyclonal antibodies, antibody fragments such as Fv, Fab, Fc, scFv and any combination thereof.

The antibodies of the present invention may originate from any source, such as any mammal, or any isolated cell capable of expressing antibodies, or any other entity capable of expressing antibodies, including bacteriophages. The antibodies of the present invention may also originate from phage display.

Additionally, the present invention relates to the use of a reagent kit as described above for assessing and/or monitoring the redox state in an individual.

Further, the present invention relates to the use of a reagent kit as described above for diagnostic and/or prognostic purposes.

Even further, the present invention relates to the use of a reagent kit as described above assessing and/or monitoring the disease state and/or disease development in an individual. In this sense, the use of the reagent kit of the invention also pertains to predict disease development, and hence serve as a kit for use in prognosis.

It is within the scope of the present invention that any use of a reagent kit as defined herein above is for use for any individual, such as a human being or an animal. In the present context the term “individual” covers any stage of organism development from a one cell zygote to fetus to fully developed organism. In an embodiment of the present invention, the use of a reagent kit as defined herein above is for use for a pregnant female and/or a fetus.

It is also within the scope of the present invention use of a reagent kit as defined herein above is for use in any disease and/or disease state, such as pregnancy associated diseases and/or complications, and/or genetic abnormalities. In an embodiment of the present invention, the use of a reagent kit as defined herein above is for use in any disease and/or disease state such as complications and/or genetic abnormalities selected from the group consisting of small-for-gestational age, preterm delivery, intrauterine growth restriction IUGR (intrauterine growth restriction), miscarriage, stillbirth, gestational hypertension, HELLP (Haemolysis, Elevated Liver Enzymes and Low Platelet count), preeclampsia, trisomy 13, trisomy 18, trisomy 21 and any combination thereof.

It should be noted that embodiments and features described in the context of one of the aspects of the present invention also apply to the other aspects of the invention.

Levels of proMBP Complexes

The normal level of the one or more proMBP complexes such as of the proMBP/AGT and/or the proMBP/PAPP-A complex in a plasma sample taken in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can in one embodiment be selected from the group consisting of from 0 nM to 50 nM, from 50 nM to 100 nm, from 100 nM to 200 nm, from 200 nM to 500 nm, from 500 nM to 750 nM, from 750 nM to 1000 nM, from 1000 nM to 1250 nM, from 1250 nM to 1500 nM, from 1500 nM to 1750 nM, from 1750 nM to 2000 nM, from 2000 nM to 2250 nM, from 2250 nM to 2500 nM, from 2500 nM to 2750 nM, from 2750 nM to 3000 nM, from 3000 nM to 3250 nM, from 3250 nM to 3500 nM, from 3500 nM to 3750 nM, from 3750 nM to 4000 nM, from 4000 nM to 4250 nM, from 4250 nM to 4500 nM, from 4500 nM to 4750 nM, from 4750 nM to 5000 nM, from 5000 nM to 5250 nM, from 5250 nM to 5500 nM, from 5500 nM to 5750 nM, from 5750 nM to 6000 nM, or any interval limited by any two of said levels.

The normal level of the one or more proMBP complexes such as proMBP/AGT and proMBP/PAPP-A in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can in one embodiment be selected from the group consisting of from 50 nM, 100 nM, 200 nM, 300 nM, 400 nM, 500 nM, 750 nM, 1000 nM, 1250 nM, 1500 nM, 1750 nM, 2000 nM, 2250 nM, 2500 nM, 2750 nM, 3000 nM, 3250 nM, 3500 nM, 3750 nM, 4000 nM, 4250 nM, 4500 nM, 4750 nM, 5000 nM, 5250 nM, 5500 nM, 5750 nM, 6000 nM, or intervals of these figures.

The threshold level of the one or more proMBP complexes such as proMBP/AGT and proMBP/PAPP-A in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can be defined as the normal level of the one or more proMBP complexes such as proMBP/AGT and proMBP/PAPP-A in the same gestation week±at least 100%, ±at least 75%, ±at least 50%, ±at least 40%, ±at least 30%, ±at least 20%, ±at least 10%, ±at least 5%, or ±at least 2% or any intervals limited by any two of said levels.

The threshold level of the one or more proMBP complexes such as proMBP/AGT and proMBP/PAPP-A in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can be defined as the normal level of the one or more proMBP complexes such as proMBP/AGT and proMBP/PAPP-A in the same gestation week±less than 100%, ±less than 75%, ±less than 50%, ±less than 40%, ±less than 30%, ±less than 20%, ±less than 10%, ±less than 5%, or ± less than 2% or any intervals limited by any two of said levels.

The threshold level of the one or more proMBP complexes such as proMBP/AGT and proMBP/PAPP-A in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can be defined as the normal level of the one or more proMBP complexes such as proMBP/AGT and proMBP/PAPP-A in the same gestation week±100%, ±75%, ±50%, ±40%, ±30%, ±20%, ±10%, ±5%, or ±2% or any intervals limited by any two of said levels.

In a preferred embodiment the present invention relates to a diagnostic and/or prognostic method comprising determination of a ratio between two different proMBP-complexes such as between proMBP/AGT and proMBP/PAPP-A.

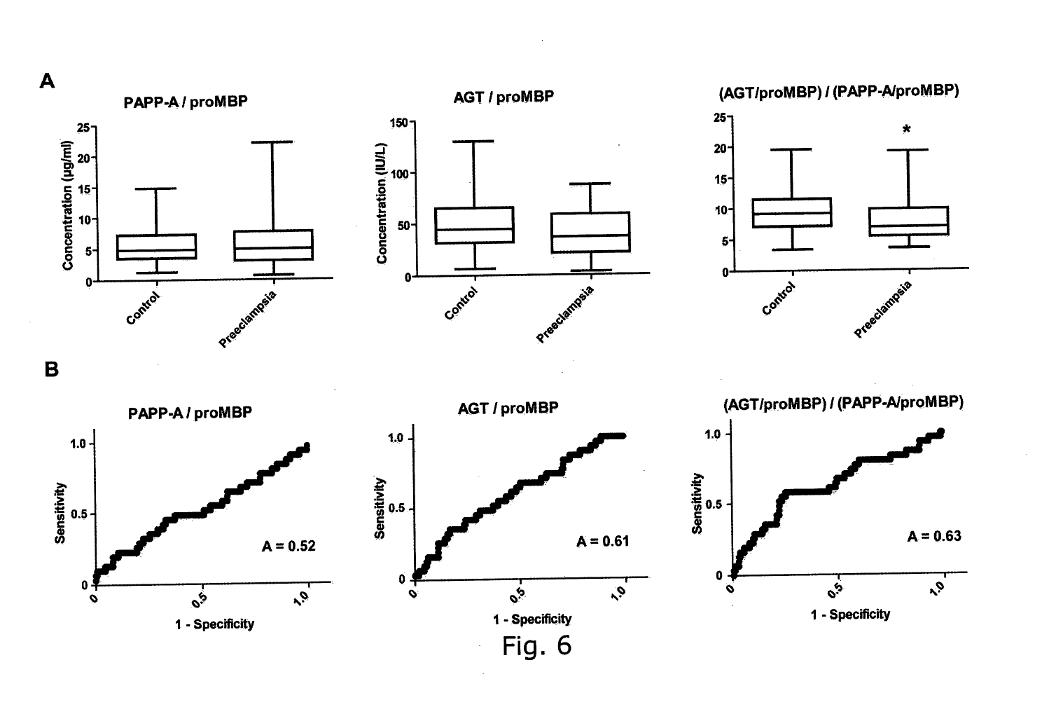

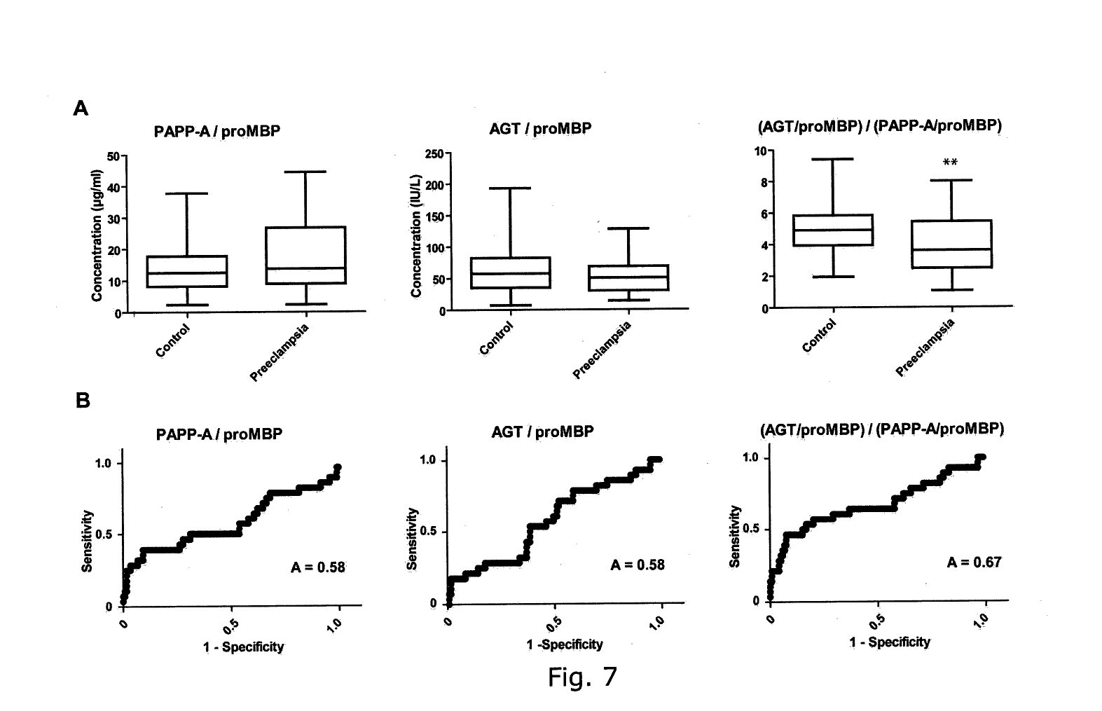

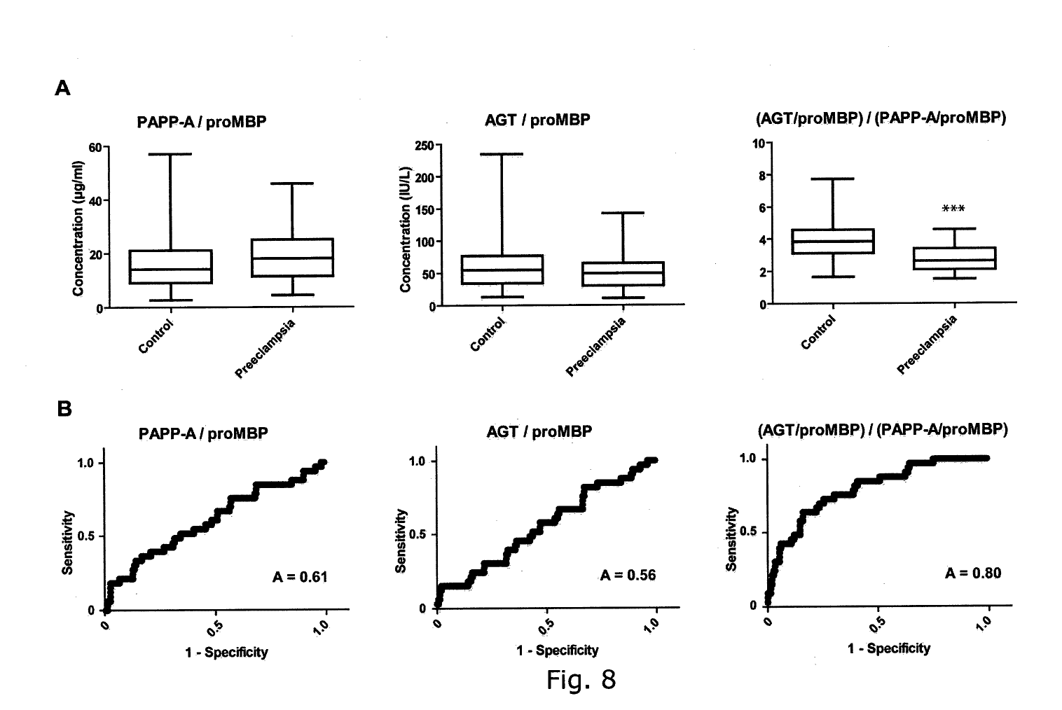

The specificity and sensitivity of a potential biomarker such as the ratio between two different proMBP-complexes such as between proMBP/AGT and proMBP/PAPP-A can be analyzed using receiver operating characteristic (ROC) curves (c.f. e.g. FIGS. 6B, 7B, and 8B). The area under ROC curves can be calculated.

The ratio between AGT/proMBP and PAPP-A/proMBP improves the diagnostic value of the individual complexes, as can be evidenced by an increased area under the ROC curve. These results suggest that the ratio between the two proMBP complexes could potentially contribute to the prediction of pregnancy complications such as preeclampsia.

In one embodiment the area under the ROC curve with respect to analysis of two proMBP complexes such as AGT/proMBP and PAPP-A/proMBP can in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 be selected from the group consisting of more than 0.5, such as more than 0.55, for example more than 0.6, such as more than 0.65, for example more than 0.7, such as more than 0.75, for example more than 0.8, such as more than 0.85, for example more than 0.9, such as more than 0.95, and for example more than 0.99.

In one embodiment the area under the ROC curve with respect to analysis of two proMBP complexes such as AGT/proMBP and PAPP-A/proMBP can in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 be selected from the group consisting of from 0.5 to 0.55, from 0.55 to 0.6, from 0.6 to 0.65, from 0.65 to 0.7, from 0.65 to 0.7, from 0.7 to 0.75, from 0.75 to 0.8, from 0.8 to 0.85, from 0.85 to 0.9, from 0.9 to 0.95, from 0.95 to 0.99 or any combination of these intervals.

In one embodiment the ratio between two different proMBP-complexes such as between proMBP/AGT and proMBP/PAPP-A in a plasma sample taken in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 from a normal individual or normal group of individuals (i.e. individuals without e.g. preeclampsia) can be selected from the group consisting of from 0 to 2, from 2 to 4, from 4 to 6, from 6 to 8, from 8 to 10, from 10 to 12, from 12 to 14, from 14 to 16, from 16 to 18, from 18 to 20, from 20 to 22, from 22 to 24, from 24 to 26, from 26 to 28, from 28 to 30, or any interval limited by any two of said levels.

In one embodiment the ratio between two different proMBP-complexes such as between proMBP/AGT and proMBP/PAPP-A in a plasma sample taken in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 from a normal individual or normal group of individuals (i.e. individuals without e.g. preeclampsia) can be less than 30, such as less than 28, for example less than 26, such as less than 24, for example less than 22, such as less than 20, for example less than 18,

such as less than 16, for example less than 14, such as less than 12, for example less than 10, such as less than 8, for example less than 6, such as less than 4, for example less than 2,
such as less than 1.

In one embodiment the ratio between two different proMBP-complexes such as between proMBP/AGT and proMBP/PAPP-A in a plasma sample taken in gestation week 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 from a normal individual or normal group of individuals (i.e. individuals without e.g. preeclampsia) can be more than 1, such as more than 2, for example more than 4, such as more than 6, for example more than 8, such as more than 10, for example more than 12, such as more than 14, for example more than 16, such as more than 18, for example more than 20, such as more than 22, for example more than 24, such as more than 26, for example more than 28, such as more than 30.

The threshold level of the ratio between two different proMBP-complexes such as between proMBP/AGT and proMBP/PAPP-A in a plasma sample taken in gestation 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can be defined as the normal level of the ratio between said two different proMBP-complexes such as between proMBP/AGT and proMBP/PAPP-A in a plasma sample taken in the same gestation week±at least 100%, ±at least 75%, ±at least 50%, ±at least 40%, ±at least 30%, ±at least 20%, ±at least 10%, ±at least 5%, or ±at least 2% or any intervals limited by any two of said levels.

The threshold level of the ratio between two different proMBP-complexes such as between proMBP/AGT and proMBP/PAPP-A in a plasma sample taken in gestation 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can be defined as the normal level of the ratio between said two different proMBP-complexes such as between proMBP/AGT and proMBP/PAPP-A in a plasma sample taken in the same gestation week±less than 100%, ±less than 75%, ±less than 50%, ±less than 40%, ±less than 30%, ±less than 20%, ±less than 10%, ±less than 5%, or ±less than 2% or any intervals limited by any two of said levels.

The threshold level of the ratio between two different proMBP-complexes such as between proMBP/AGT and proMBP/PAPP-A in a plasma sample taken in gestation 18 and/or week 19 and/or week 20 and/or week 21 and/or week 22 and/or week 23 and/or week 24 and/or week 25 and/or week 26 and/or week 27 and/or week 28 and/or week 29 and/or week 30 and/or week 31 and/or week 32 and/or week 33 and/or week 34 and/or week 35 and/or week 36 and/or week 37 and/or week 38 and/or week 39 and/or week 40 and/or week 41 and/or week 42 can be defined as the normal level of the ratio between said two different proMBP-complexes such as between proMBP/AGT and proMBP/PAPP-A in a plasma sample taken in the same gestation week±100%, ±75%, ±50%, ±40%, ±30%, ±20%, ±10%, ±5%, or ±2% or any intervals limited by any two of said levels.

### Sampling Time During Gestation

The sample to be analysed can be obtained from the individual any time during pregnancy such as in gestation week 18, 19, 20, 21, 22, 23, 24, 25, 26, 27, 28, 29, 30, 31, 32, 33, 34, 35, 36, 37, 38, 39, 40, 41 and/or 42.

One or more samples from the same individual can be obtained and analysed such as 2, 3, 4, 5, 6, 7, 8, 9, 10 or more than 10 samples from the same or preferably from different gestation weeks.

The first analysis of the one or more proMBP complexes and/or the one or more secondary markers can be performed on a sample derived from gestation week 18, 19, 20, 21, 22, 23, 24, 25, 26, 27, 28, 29, 30, 31, 32, 33, 34, 35, 36, 37, 38, 39, 40, 41 and/or 42.

The second analysis of the one or more proMBP complexes and/or the one or more secondary markers can be performed on a sample derived from gestation week 18, 19, 20, 21, 22, 23, 24, 25, 26, 27, 28, 29, 30, 31, 32, 33, 34, 35, 36, 37, 38, 39, 40, 41 and/or 42.

The third analysis of one or more proMBP complexes and/or the one or more secondary markers can be performed on a sample derived from gestation week 17, 18, 19, 20, 21, 22, 23, 24, 25, 26, 27, 28, 29, 30, 31, 32, 33, 34, 35, 36, 37, 38, 39, 40, 41 and/or 42.

The fourth analysis of one or more proMBP complexes and/or the one or more secondary markers can be performed on a sample derived from gestation week 18, 19, 20, 21, 22, 23, 24, 25, 26, 27, 28, 29, 30, 31, 32, 33, 34, 35, 36, 37, 38, 39, 40, 41 and/or 42.

The fifth analysis of one or more proMBP complexes and/or the one or more secondary markers can be performed on a sample derived from gestation week 17, 18, 19, 20, 21, 22, 23, 24, 25, 26, 27, 28, 29, 30, 31, 32, 33, 34, 35, 36, 37, 38, 39, 40, 41 and/or 42.

The sixth or any further analysis of one or more proMBP complexes and/or the one or more secondary markers can be performed on a sample derived from gestation week 17, 18, 19, 20, 21, 22, 23, 24, 25, 26, 27, 28, 29, 30, 31, 32, 33, 34, 35, 36, 37, 38, 39, 40, 41 and/or 42.

In one embodiment more than one analysis is performed if the first or a subsequent analysis was inconclusive with respect to if the subject has a pregnant associated disease such as preeclampsia or has an increased risk of developing a pregnant associated disease such as preeclampsia.

In one embodiment more than one analysis is performed if the first or a subsequent analysis showed that the individual has an increased risk of developing a pregnant associated disease such as preeclampsia.

The gestation time of the individual and the gestation time of the population used to determine the threshold value or normal value are preferably similar such as within three weeks of each other, within two weeks of each other, or within one week of each other.

### Secondary Markers

The above-described method for diagnosing and/or prognosing comprising measurements of two or more proMBP-complexes can further comprise:
(i) determining a level in the subject of a secondary marker for a pregnancy associated disease or complication such as for preeclampsia; and
(ii) comparing said secondary marker level to a standard level for said secondary marker;
   wherein if said secondary marker level is different from—i.e. either greater than or less than—said secondary marker standard level the subject is identified as more likely to have preeclampsia or to develop preeclampsia, and if said secondary marker level is not greater than or less than said secondary marker standard level the subject is identified as less likely to have preeclampsia or to develop preeclampsia. Accordingly, use of one or more secondary marker(s) in combination with the above described method for diagnosing and/or prognosing can improve the accuracy of the diagnostic and/or prognostic method.

The blood pressure or urinary protein content of the individual can also be determined and this analysis can be taken into account when preeclampsia is diagnosed or prognosed.

In one embodiment the present invention relates to methods of assessing, predicting and diagnosing preeclampsia wherein the level of proMBP complexes and one or more further secondary markers associated with preeclampsia is detected or monitored. The number of secondary markers associated with preeclampsia can be 1, 2, 3, 4, 5, 6, 7, 8, 9, 10 or more than 10 different secondary markers.

The level of the one or more secondary markers can be measured at the same gestational age as the level of the proMBP complexes is measured or alternatively the level of the secondary marker can be determined at a different gestational age than where the proMBP complex levels is determined.

The level of the one or more secondary markers can be measured in the same diagnostic assay as where the level of the proMBP complexes is measured or alternatively the level of the secondary marker can be determined in a different diagnostic assay than where the proMBP complex levels are determined.

The level of the one or more secondary markers can be measured in the same type of sample—such as e.g. a serum, a blood sample, a placenta biopsy sample or a urine sample—as the sample wherein the level of the proMBP complexes is measured or alternatively the level of the secondary marker can be measured in a different type of sample than the sample wherein the proMBP complex levels is determined.

The one or more secondary markers associated with preeclampsia can be one or more secondary markers described in one or more of the US patent applications selected from the group consisting of US 20120189632, US 20120142559, US20120135427, US 20120101021, US 20120040371, US 20110318809, US20110280863, US 20110269136, US20110171650, US 20110104107, US20110059904, US 20100323352, US 20100291612, US20100227342, US20100190181, US 20100113286, US 20100105070, US 20100017143, US20100016173, US 20080233583 and US 20060252068.

The content of US 20120189632, US 20120142559, US 20120135427, US 20120101021, US 20120040371, US 20110318809, US 20110280863, US 20110269136, US20110171650, US 20110104107, US 20110059904, US 20100323352, US 20100291612, US20100227342, US 20100190181, US 20100113286, US 20100105070, US 20100017143, US20100016173, US 20080233583 and US 20060252068 is incorporated in their entireties herein.

In another embodiment the one or more secondary markers associated with preeclampsia can be one or more secondary markers selected from the group consisting of ABLIM2, ACACA, ACOT8, ASCL2, AHSG, ALDH1A2, ALS2CL, ANXA13, APC, AQP2, ARFGEF2, ART1, ASCL2, ATP7B, AXIN1, BDKRB2, BICD1, C3, C4BPA, C4orf10, C6orf142, CCK, CD52, CDH15, CENTG3, CFHR1, CHERP, CHF, CHRDL1, CHST6, CFHR1, CLASP2, CLCN7, CLDN6, CTAG2, COL5A1, COL9A3, CPM, CRI, CRYBB1, CUL1, CYP4A11, CXCL9, DEPDC7, DHRS2, DLGAP1, DPYSL4, ELL2, EGLN3, EGR1, EPAS1, F11R, F2R, FKBP1A, FLRT2, FLT4, FN1, FOS, FOSB, FPRL2, FSTL3, HSP3, FUT6, FZD5, GATA1, GNG4, GNG7, GNLY, GRP109B, GTPBP2, GZMB, HBE1, HBZ, HCFC1, HEXA, HLA-DQA1, HOXB7, HPS3, HTR2B, HYDIN, IGFBP1, IGKC, IKZF1, IL15, IL1B, IL1RL1, IL2RB, ISG20, ITGB6, KCNH2, KCNIP3, KCNQ1, KISS1R, KLRC2, KRT14, LAIR2, LIPH, LOC440157, LRAP, LSS, LTBR, MAGEB6, MAOB, MAP2K7, MGA, MMD, MMP12, MSR1, MUC4, MUC15, MYL9, NDP, NDUFV2, NINJ2, NKX2-5, NMNAT3, NOG, NTN1, OPRL1, OXGR1, P4HA3, PACAP, PAEP, PDE4C, PDE9A, PITPNC1, PLAC8, PRDM1, PRG2, PRL, PSCD2, PTPRN, PTPRS, RAB12, RAD52, RBP4, REST, RHD, RORB, RUFY3, S100A8, S100A12, SART3, SCARA5, SDHAL1, SEMA3C, SERPINA3, SEZ6L, SLC13A4, SLC13A5, SLC2A3, SLC16A6, SLCO4A1, SPDEF, SPG20, SPOCK1, SPRR2B, SRF, SSTR1, ST6GALNAC4, STXBP2, THBS4, TMC4, TNRC9, TPM1, TRA@, TRIM3, UGGT2B7, WNT10B, WT1, ZP3, tenascin-X (TNXB), basement membrane-specific heparan sulfate proteoglycan core protein (HSPG2), cell surface glycoprotein (CD146, MUC18, MCAM), phosphatidylinositol-glycan-specific phospholipase D (GPLD1), collagen alpha-3(VI) chain (COL6A3), Kunitz-type protease inhibitor 1 (SPINT1), hepatocyte growth factor-like protein (MST1), probable G-protein coupled receptor 126 (GPR126), intercellular adhesion molecule 3 (ICAM3), C-reactive protein (CRP), disintegrin and metalloproteinase domain-containing protein 12 (ADAM12), phosphatidylcholine-sterol acyltransferase (LCAT), roundabout homolog 4 (ROBO4), ectonucleotide pyrophosphatase/phosphodiesterase family member 2 (ENPP2), insulin-like growth factor-binding protein complex acid labile subunit (IGFALS), SEPP1, s-Endoglin (ENG or s-ENG), quiescin Q6 (QSOX1), peroxiredoxin-2 (PRDX2), blood glucose level, body mass index (BMI), ‘high density lipoprotein level’, ‘ratio of total cholesterol to high density lipoprotein’, metabolic syndrome, triglycerides level, vascular endothelial growth factor receptor 3 (FLT4), lysosomal Pro-X carboxypeptidase (PRCP), peroxiredoxin-1 (PRDX1), leucyl-cystinyl aminopeptidase (LNPEP), protein S100-A9 (S100A9), SEPP1, ENG, QSOX1, PRDX2, PIGF, Activin A, P-Selectin, angiogenic factors, VEGF, PIGF, sFlt-1, Chaperonin, ER-60 protease, Alpha-enolase, Isocitrate dehydrogenase I, Aldehyde reductase, Chain B Fidarestat Bonded to human Aldose reductase, Voltage-dependent anion channel 1, Nuclear chloride channel, Chain H Cathepsin D, Phosphoglycerate mutase I, Endoplasmatic reticulum protein, PSMA2 protein, Glutathione S-transferase, Ig heavy chain v region, Smooth muscle myosin alkali light chain, Fatty acid binding protein, supramolecular aggregate of misfolded proteins that is associated with (is a causative factor in the pathology of) preeclampsia in e.g. a urine sample or placenta tissue sample, supramolecular aggregate of misfolded proteins comprising serpina-1 (alpha-1 antitrypsin) or a fragment of serpina-1, supramolecular aggregate further comprising at least one of ceruloplasmin, heavy-chain IgG, light-chain IgG and interferon inducible protein 6-16 (IFI6), placental chondroitin 4-O-sulfotransferase 1 (C4ST), chondroitin 6-sulfotransferase (C6S), heparan sulfate 6-O-sulfotransferase 1 (HS6S), dermatan/chondroitin sulfate 2-sulfotransferase (CS-20ST), uronic acid-2-sulfate (UA2S), glycosaminoglycan (GAG) synthesis regulatory enzyme from a placental tissue, methylation of Maspin gene (differentially methylated in fetal DNA and in maternal DNA), calcyclin, expression level of calcyclin in chorionic villi, levels of angiogenic factors, specifically VEGF, PIGF and sFlt-1 in urine samples, histidine and ketone bodies, fatty acid binding protein 4 (FABP4), Peroxiredoxin 6, enoyl-CoA hydratase (ECHS1), human olacental lactogen, DELTA3,5-DELTA2,4-dienoyl-CoA isomerase (ECH1), Per6, heat shock protein beta-1 (HSP27), stathmin, FABP4, ECHS1, ECH1, heat shock protein E-backward-1, lipocortin, prostaglandin dehydrogenase 1, proliferation-associated protein 2G4, placental growth hormone (chorionic sommatomammotropin hormone (CSH1), estradiol 17-beta dehydrogenase, macrophage capping protein, levels of free haemoglobin, particularly free fetal haemoglobin, endothelin, soluble forms-like tyrosine kinase-1 (sFlt-1), angiotensin-II, Alpha-1 B-glycoprotein, -Actin, Apolipoprotein B-100, Apolipoprotein C-Il, Apohpoprotein C-III, C4b-binding protein beta chain, Cathepsin D, Choriogonadotropin subunit beta, Cholinesterase, Chorionic somatomammotropin hormone, Cystatin-C, Endoglin, Coagulation factor XI, Coagulation factor VII, Fibronectin, Filamin-A, Heparin cofactor 2, Hepatocyte growth factor-like protein, Histidine-rich glycoprotein, Insulin-like growth factor-binding protein 2, Laminin subunit beta-1, Lipopolysaccharide-binding protein, Matrix metalloproteinase-9, Plastin-2, Profilin-1, Pregnancy-specific beta-1-glycoprotein, Receptor-type tyrosine-protein phosphatase gamma, Pregnancy zone protein, Plasma retinol-binding protein, SH3 domain-binding glutamic acid-rich-like protein 3, Transgehn-2, Talin-1, Tropomyosin alpha-4 chain, Vasorin, Vascular endothelial growth factor receptor 3, Vinculin, von Willebrand factor, Pappalysin-2, Alpha-2-antiplasmin, Actin, Afamin, Antithrombin-III, Apolipoprotein A-Il, Attractin, Beta-2-microglobulin, Transforming growth factor-beta-induced protein ig-h3, C4b-binding protein alpha chain, Carboxypeptidase B2, Complement factor D, Cartilage acidic protein 1, Dopamine beta-hydroxylase, Coagulation factor XIII B chain, Fibrinogen alpha chain, Rho GDP-dissociation inhibitor 2, Platelet glycoprotein Ib alpha chain, Haptoglobin-related protein, Platelet basic protein, Tubulin beta-1 chain, Thymosin beta-4, Vascular cell adhesion protein 1, Zinc-alpha-2-glycoprotein, Alpha-2-macroglobulin, Pappalysin-1, C-reactive protein, Serum amyloid P-component, Complement factor H-related 5, Protein piccolo, Xaa-Pro dipeptidase, Protein bassoon, Dystroglycan, Catalase, Carbonic anhydrase 1, Intracellular adhesion molecule 1, Serotransferrin, Galectin-3-binding protein, Peroxiredoxin-2, Biphosphoglycerate mutase, Corticosteroid-binding globulin, Carbonic anhydrase 2, Adenomatous polyposis coli protein, Latent-transforming growth factor beta-binding, Coagulation factor IX, Hepatocyte growth factor activator, Complement C1q subcomponent subunit C, Complement C1q subcomponent subunit B, Cartilage oligomeric matrix protein, gamma-enteric smooth muscle, Mast/stem cell growth factor receptor, Platelet glycoprotein V, Roundabout homolog 4, Extracellular matrix protein 1, Complement C1q subcomponent subunit A, Phospholipid transfer protein, ADAMTS-13, Plasma protease C1 inhibitor, Apolipoprotein F, Noelin, Low affinity immunoglobulin gamma Fc region receptor, CD44 antigen, Macrophage mannose receptor 1, Fibrinogen beta chain, Membrane copper amine oxidase, Alpha-1-acid glycoprotein 1, Cadherin-5, Fructose-biphosphate aldolase A, Probable G-protein couple receptor 126, 14-3-3 protein zeta/delta, Cofilin-1, Glycealdehyde-3-phosphate dehydrogenase, N-acetylglucosamine-1-phosphotransferase subunit gamma, Alpha-actinin-1, Phosphoglycerate mutase 1, Term-like transcript 1 protein, Glutathione S-transferase P, Leucyl-cystinyl aminopeptidase, Adenylyl cyclase-associated protein 1, Peptidyl-prolyl cic-trans isomerase A, Transketolase, Phosphoglycerate kinase 1, Leptin, cortocotropin releasing hormone, inhibit Beta A, chorionic gonadotropin beta polypeptide, NA, Fms-related tyrosine kinase 1 (VEGFR), sialic acid binding Ig-like lectin 6, luteinizing hormone beta polypeptide, B-cell CLL/lymphoma 6, inhibin alpha, pappalysin 2, endoglin (Osler-Rendu-Weber syndrome 1), Sperm associated antigen 4, Retinol dehydrogenase 13 (all-trans and 9-cis), glucosidase beta acid, protease serine, 11 (IGF binding), SHS multiple domains 1, Solute carrier family 2, synapse defective 1, Rho GTPase, scavenger receptor class B member 1, solute carrier organic anion, mannosidase alpha class 1C member 1, frizzled homolog 10, KIAA1211 protein, UDP-Gal:betaGlcNAc, BTG family, member 2, hypothetical protein MGC17839, potassium channel subfamily K member 3, chromosome 1 open reading frame 139, glutathione S-transferase A3, fibrillin 2, carbonic anhydrase X, ankyrin repeat and SOCS box-containing 2, hydroxysteroid (17-beta), SIPA1L1, VDR, SASH1, PKD1L2, GLRX, DDR1, ARP3BETA, TIF1, LRRC1, FCN3, PHYHIP, TGOLN2, SREBF1, TTC17, RPL10, EVER1, ZC3HDC6, MGC17839, HCA112, MAPK8, KIAA1211, RASSF6, BTG2, FLJ90586, SLC26A2, MGC17839, FLJ23091, GSTA3, LEPREL1, FBN2, ASB2, CLDN1, ANTXR1, HSD17B1, TRIP10, IL1A, CGA, SYDE1, EPHA1, FLJ90575, B4GALT5, KCNK3, CA10, ADORA2B, MMP12, GKN1, C1orf139, IGFBP3, ABP1, FN1, INHBA, SLC21A2, SIGLEC6, KIAA0992, TIMP3, LEP, LPL, dehydrogenase 1, anthrax toxin receptor 1, LEP, CRH, LPL, INHBA, LPL, CRH, FLT1, CGB, CGB5, CGB7, FABP4, BCL6, INHBA, SIGLEC6, LHB, INHA, FSTL3, ADFP, TFPI, ERO1L, ENG, MME, CALM1, KIAA1102, LTF, RDH13, MBD2, SASH1, KIF2, PPL, NDRG1, SPAG4, KIAA1102, TPBG, MBD2, SLCO4A1, HA-1, LGALS8, BTNL9, EPS8L1, TFPI, C20orf38, CBLB, BCL6, HRASLS3, CALM1, SH3BP5, KIAA1984, SH3BP5, GBA, GBAP, EPS8L1, SH3MD1, EPS8L1, SASH1, HIG2, SLC6A8, FLJ143855, LRRC1, MAST4, FZD10, SLCO2A1, MTMR4, HSA9761, 7h3, PRSS11, PIK3AP1, GREM2, LGALS8, MAN1C1, SLC2A14, TUBA1, NEK11, TXNDC4, CSNK2A2, EFHD1, EBI3, KIF2, DUSP1, SCARB1, ADAM12, HEY1, and LOC255743.

The level of the one or more secondary markers can e.g. be determined at the protein or nucleotide level. The activity of the one or more secondary markers can also be measured.

### Experimental Procedures and Conditions

### Plasma and Serum Samples

Third trimester EDTA-plasma was obtained from seven anonymous normotensive pregnant women in the third trimester at Holbæk Hospital (primary hospital), Denmark. In addition, paired EDTA-plasma and serum samples were collected from three anonymous normotensive pregnant women in the third trimester. All samples were separated, immediately placed on ice, and frozen at −20° C. Term pregnancy blood from anonymous pregnant women was collected at Skejby Hospital, Denmark, allowed to form serum at RT over 5-10 h, and stored for several months at −20° C. For some experiments, samples of third trimester plasma and serum were incubated for 24 h at RT in the absence or presence of 50 mM iodoacetamide.

### Western Blotting

AGT specific Western blotting was performed following separation of proteins by 10-20% (Tris/glycine) non-reducing or reducing SDS-PAGE. Proteins were blotted onto a polyvinylidene difluoride membrane (Millipore) and blocked in 2% skimmed milk powder diluted in TST (50 mM Tris, 500 mM sodium chloride, and 0.1% Tween 20, pH 9.0). The membrane was then washed in TST and incubated overnight (ON) at 4° C. with a mouse anti-AGT monoclonal antibody (mAb) (F8A2) diluted to 1 μg/ml in TST containing 2% skimmed milk powder. Thereafter, the membrane was washed and incubated for 0.5 h at RT with peroxidase-conjugated rabbit anti-mouse antibodies (P0260, Dako) diluted 1:2000 in TST containing 2% skimmed milk powder. The blots were developed using enhanced chemiluminescence (ECL, Amersham Biosciences) and visualized using X-ray film. Detection of proMBP was performed similarly. Mouse anti-proMBP mAbs 234-10, diluted to 1 μg/ml, was used to detect non-reduced proMBP. Dithiothreitol (DTT) was used as reducing agent.

### Enzyme-Linked Immunosorbent Assay (ELISA)

Protein concentrations were measured by sandwich ELISAs performed in Maxisorp polystyrene microtiter plates (Nunc). Coating antibodies were diluted in coating buffer (0.1 M sodium bicarbonate, pH 9:8), and wells were blocked with 2% BSA in PBS (20 mM sodium hydrogen phosphate, 150 mM sodium chloride, pH 7.4). Detecting antibodies, samples, and calibrators were diluted in PBS-T (0.01% Tween-20 in PBS) supplemented with 1% BSA. Washing was carried out with PBS-T. AGT specific ELISA: A mouse anti-AGT mAb (F8A2), diluted to 2 μg/ml, was used for capture. Detection was performed using polyclonal chicken anti-AGT (SSI 233), diluted 1:1000, followed by peroxidase-conjugated rabbit anti-chicken IgY (A9046, Sigma-Aldrich). ProMBP specific ELISA: Polyclonal rabbit anti-PAPP-A/pro-MBP, diluted to 5 μg/ml, was used for capture. Detection was performed using mouse anti-proMBP mAb (234-10), diluted to 1 μg/ml, followed by peroxidase-conjugated rabbit anti-mouse IgG (P0260, Dako). PAPP-A specific ELISA: Polyclonal rabbit anti-PAPP-A/proMBP, diluted to 5 μg/ml, was used for capture. Detection was performed using a mouse anti-PAPP-A mAb (234-5), diluted to 1 μg/ml, followed by peroxidase-conjugated anti-mouse IgG (P0260, Dako). ProMBP/AGT specific ELISA: Mouse anti-AGT mAb (F8A2), diluted to 2 μg/ml, was used for capture. Detection was performed using a biotinylated mouse anti-proMBP mAb (234-10), diluted to 1 μg/ml, followed by peroxidase-conjugated avidine (P0347, Dako). In this assay, 0.8 M sodium chloride was added to buffers for washing and dilution. ProMBP/PAPP-A specific ELISA: Mouse anti-PAPP-A mAb (234-5), diluted to 2 μg/ml, was used for capture. Detection was performed using a biotinylated mouse anti-proMBP mAb (234-10), diluted to 1 μg/ml, followed by peroxidase-conjugated avidine (P0347, Dako). In this assay, 0.8 M sodium chloride was added to buffers for washing and dilution. Dilution series of third trimester pregnancy plasma was used to establish standard curves for measurement in blood samples. Concentrations were measured in arbitrary units. One arbitrary unit is equivalent to the concentration of the protein in the non-diluted calibrator. For measurement of recombinant proteins, the assays were calibrated with purified proMBP/PAPP-A complex or immunoaffinity (F8A2) purified recombinant AGT, quantified by amino acid analysis.

### Size-Exclusion Chromatography

Plasma from seven normotensive pregnant women was thawed on ice, pooled, and diluted 20 times in running buffer (20 mM Tris, 150 mM sodium chloride, 0.02% Tween-20, pH 7.4). To separate monomeric AGT from HMW AGT, size-exclusion chromatography was performed on a column (330 ml) packed with Sephacryl S-200 (Amersham Biosciences) and equilibrated in the running buffer. The flow rate was 0.75 ml/min, and fractions of 3 ml were collected. Individual fractions were analyzed by Western blotting and ELISA.

### Immunodepletion

For depletion of proMBP, chromatographic fractions were incubated ON at 4° C. with a mouse anti-proMBP mAb (PM-5A) immobilized to CNBr-activated Sepharose 4B (GE Healthcare). Immunodepletion of proMBP was verified using the proMBP specific ELISA, and samples were analyzed by AGT specific ELISA and AGT specific Western blotting following reducing SDS-PAGE. For depletion of PAPP-A and AGT, chromatographic fractions were incubated with mouse anti-PAPP-A mAb (234-5) and anti-AGT mAb (F8A2) immobilized to CNBr-activated Sepharose 4B. Controls were carried out by incubation with CNBr-activated Sepharose 4B coupled with an irrelevant mouse monoclonal antibody (anti-c-myc, 9E10). For depletion of C3d in serum, samples were incubated ON at 4° C. with protein-G Sepharose (GE Healthcare) in the absence or presence of rabbit anti-C3d polyclonal antibodies (A063, Dako), and analyzed by proMBP specific Western blotting.

### Cell Culture and Transfection

Human embryonic kidney 293T cells (293tsA1609neo) were maintained in high glucose Dulbecco's modified Eagle's medium supplemented with 10% fetal bovine serum, 2 mM glutamine, nonessential amino acids, and gentamicin (Invitrogen). Cells were plated onto 6-cm culture dishes and transiently transfected 18 h later by calcium phosphate co-precipitation using 10 μg of plasmid DNA prepared by GenElute Plasmid Miniprep kit (Sigma-Aldrich). The cells were transfected with either PAPP-A cDNA, cDNA encoding proMBP, mutated variants of proMBP (proMBP(C51S) and proMBP(C169S), or AGT cDNA, and the culture medium was harvested after 48 h. cDNA encoding AGT was kindly provided by Professor Xavier Jeunemaitre, and transferred from the original pECE vector to pcDNA3.1(+)/myc-His (Invitrogen) using the restriction enzymes XbaI and HindIII. Protein concentrations in culture media were measured by ELISA, as specified above.

### In Vitro Complex Formation

Recombinant PAPP-A or proMBP, were mixed to final subunit concentrations of 20 nM and incubated at 37° C. to allow the covalent complex to form in the presence 0-240 nM recombinant AGT. The experiment was carried out in the presence of 30-180 μM GSH. The amount of proMBP/PAPP-A complex formed was measured over time using the proMBP/PAPP-A specific ELISA.

### EXAMPLES

### Example 1

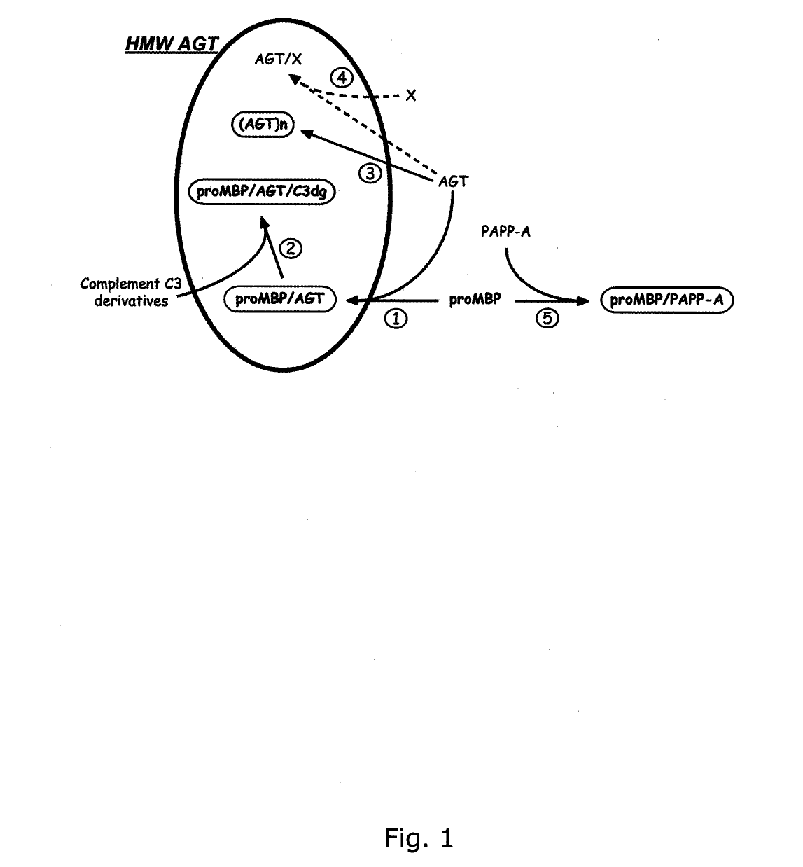

### FIG. 1

Network of protein complexes present in the circulation of pregnant women. Solid lines denote formation of complexes reported in the literature; dashed lines indicate formation of hypothetical complexes. Protein complexes potentially contributing to the HMW fraction of AGT are within the grey circle. The covalent 2:2 proMBP/AGT complex resulting from reaction 1, and its further complex with C3dg following reaction 2, have both been detected in the circulation. Multimerization of AGT (reaction 3) is known to occur in vitro, and has been speculated to occur in pregnant women. Reaction 4 indicates the possibility that unknown proteins may react with uncomplexed AGT. ProMBP, thus potentially involved in several different AGT complexes, is known to also form the 2:2 proMBP/PAPP-A complex (reaction 5).

### Example 2

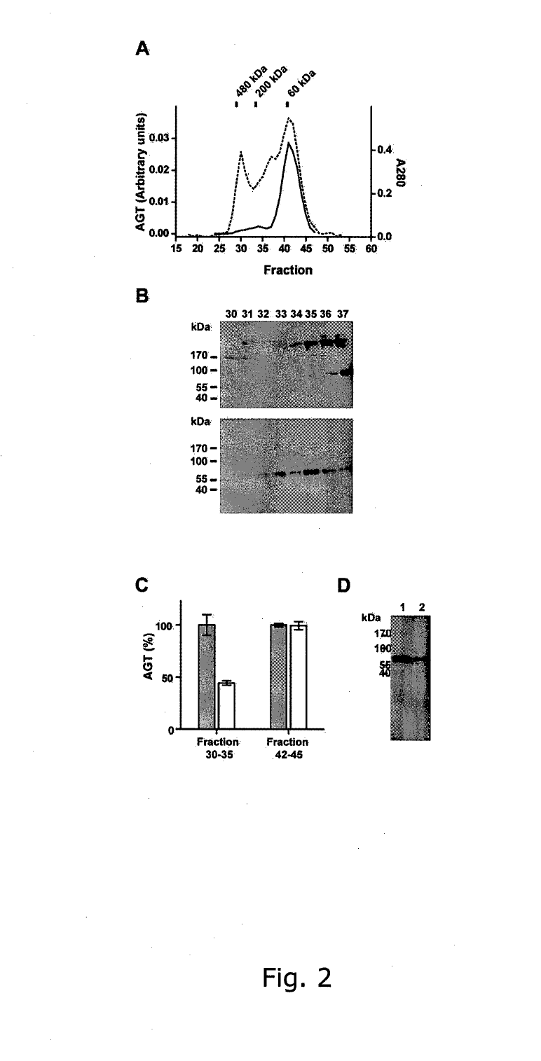

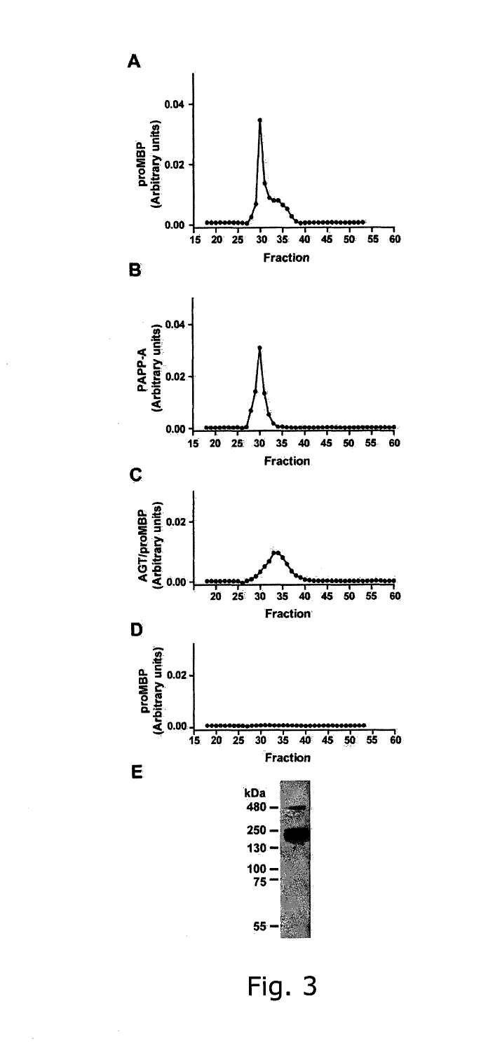

### FIG. 2

ProMBP is a major constituent of the high-molecular weight fraction of angiotensinogen in late pregnancy plasma. A, separation of AGT species in freshly obtained third trimester plasma by size-exclusion chromatography (Sephacryl S-200). Elution profile of total protein (dashed line, absorption at 280 nm) and AGT antigen (solid line) are shown. The latter was determined by an AGT specific ELISA. B, Western blot of fractions 30-37 using an AGT specific mAb following non-reducing (upper panel) and reducing (lower panel) 10-20% SDS-PAGE. C, fractions 30-35 (HMW AGT) and fractions 42-45 (monomeric AGT) were pooled and analyzed by an AGT specific ELISA before (grey bars) and after (white bars) immunodepletion of proMBP. The data shown are the means+/− standard deviations of three independent experiments. D, Western blot using an AGT specific mAb following reducing SDS-PAGE of fractions 30-35 before (lane 1) and after (lane 2) immunodepletion of proMBP. Plasma from seven normotensive pregnant women in the third trimester was obtained, placed immediately on ice, and frozen.

In summary, a pool of all plasma samples was subjected to size-exclusion chromatography, and individual fractions were analyzed using an AGT specific ELISA (FIG. 2A). AGT antigen eluted as a major peak corresponding to monomeric AGT (fractions 37-46) and a smaller, broad peak of HMW AGT (fractions 29-38). The total amount of AGT in the HMW fractions relative to monomeric AGT is in agreement with previous studies, in which the AGT concentration was measured by quantification of angiotensin 1 generation after renin cleavage.

Western blotting following non-reducing SDS-PAGE of the HMW fractions revealed several AGT reactive species (FIG. 2B, upper panel). Upon reduction of the material in these fractions, AGT migrated as a monomer of the expected 60 kDa (FIG. 2B, lower panel), demonstrating that it is present in disulfide linked HMW complexes.

Fractions 30-35, containing HMW AGT complexes, were pooled and immunodepleted for proMBP. This caused more than a 50% reduction of AGT antigen, as measured by ELISA (FIG. 2C). Western blotting following reducing SDS-PAGE of the depleted sample also showed a dramatic reduction in the amount of AGT (FIG. 2D), confirming that proMBP is a major constituent of HMW AGT in late pregnancy plasma.

### Example 3

### FIG. 3

ProMBP is disulfide linked to PAPP-A or AGT in late pregnancy plasma. Freshly obtained third trimester pregnancy plasma was separated by size-exclusion chromatography (Sephacryl S-200). Individual fractions were analyzed by ELISA for their content of proMBP (A), PAPP-A (B), and proMBP/AGT (C). Following combined immunodepletion of PAPP-A and AGT, no proMBP could be detected by ELISA in individual fractions (D). E, proMBP specific Western blot following non-reducing 10-20% SDS-PAGE of unfractionated, freshly obtained late pregnancy plasma.

In summary, measurement of proMBP in individual chromatographic fractions by ELISA showed an elution profile of two overlapping peaks (FIG. 3A). Importantly, this experiment reveals that proMBP does not occur as an uncomplexed monomer in the circulation, expected to elute after fraction 45. Measurement of PAPP-A (FIG. 3B) indicates the presence of the proMBP/PAPP-A complex corresponding to the position of the left peak of FIG. 3A, and measurement of the proMBP/AGT complex (FIG. 3C) indicates the presence of this complex (or proMBP/AGT/C3dg) corresponding to the broader, right peak. Upon immunodepletion of both AGT and PAPP-A in individual fractions, proMBP antigen could not be detected (FIG. 3D). Together, these experiments demonstrate that all of proMBP circulating during pregnancy is present in protein complexes with either PAPP-A or AGT, and that no other complexes of proMBP (FIG. 1, reaction 6) are present. ProMBP specific Western blotting (FIG. 3E) showed two bands, supporting this interpretation. The molecular weight of these bands correspond to the presence of the 2:2 proMBP/PAPP-A complex (480 kDa), and the 2:2 proMBP/AGT complex (200 kDa), both previously isolated from pregnancy plasma. Interestingly, this experiment did not reveal the presence of the 300 kDa 2:2:2 proMBP/AGT/C3dg complex (FIG. 1, reaction 2).

### Example 4

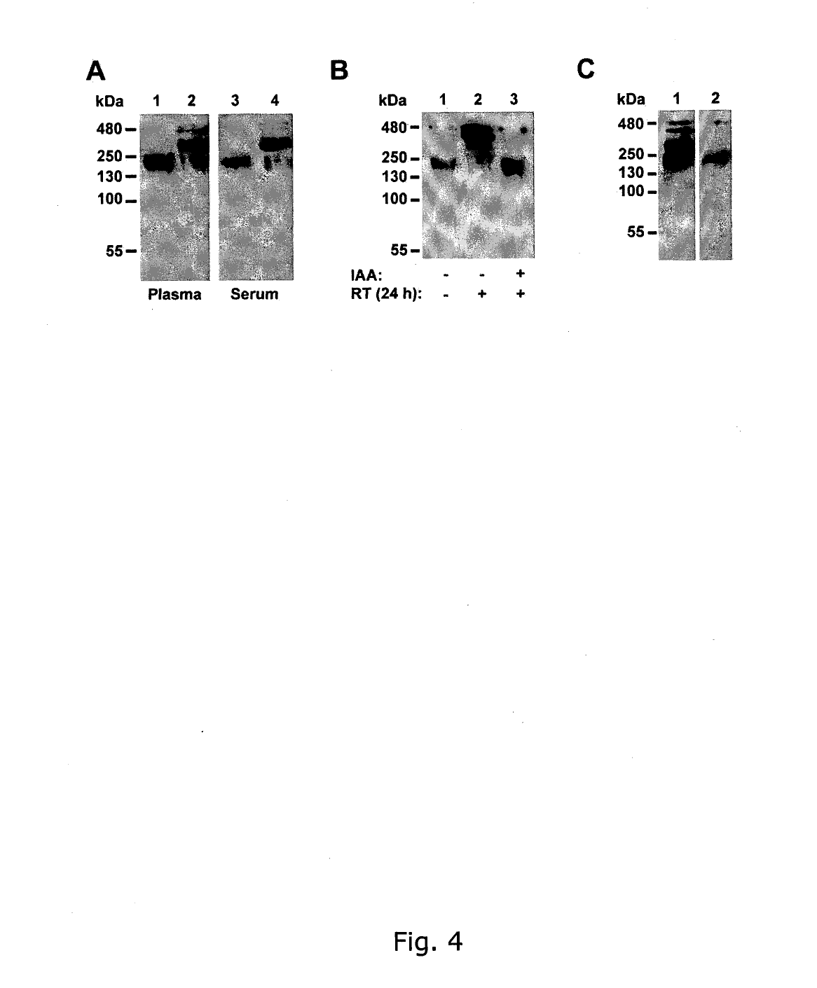

### FIG. 4

Complement C3 containing complexes form as a consequence of post-sampling events in late pregnancy plasma and serum. The results are presented in FIG. 4. A, proMBP specific Western blot following non-reducing 10-20% SDS-PAGE of paired third trimester samples of plasma (lanes 1 and 2) and serum (lanes 3 and 4) incubated at room temperature (RT) for 0 hours (lanes 1 and 3), or for 24 hours (lanes 2 and 4). B, proMBP specific Western blot following non-reducing 10-20% SDS-PAGE of third trimester serum incubated for 0 hours (lane 1) or 24 hours at RT in the absence (lane 2) or presence (lane 3) of alkylating agent (iodoaceteamide, IAA). C, proMBP specific Western blot following non-reducing 10-20% SDS-PAGE of term pregnancy serum (lane 1), and C3d depleted term pregnancy serum (lane 2).

In summary, the absence of the 2:2:2 proMBP/AGT/C3dg complex in freshly obtained plasma samples indicates that post-sampling events may lead to its formation. Therefore, plasma samples were incubated for 24 hours at room temperature and analyzed by proMBP specific Western blotting following non-reducing SDS-PAGE. Dramatic changes in the molecular weight of the proMBP species were observed (FIG. 4A, lanes 1 and 2). A similar result was obtained with freshly obtained pregnancy serum (FIG. 4A, lanes 3 and 4). However, the observed shift in molecular weight was inhibited by the presence of alkylating reagent (FIG. 4B). Therefore, incubation, not processes of blood coagulation, results in these changes towards higher molecular weight, which occur as a consequence of disulfide bond formation. It is therefore reasonable to think that the formed complexes may involve complement C3dg. To allow analysis of putative C3dg containing complexes, serum formed at room temperature over several hours from term pregnancy blood was analyzed by Western blotting following non-reducing SDS-PAGE. In addition to the bands corresponding to the proMBP/PAPP-A complex and the proMBP/AGT complex, several additional proMBP-reactive bands were observed (FIG. 4C, lane 1). However, following immunodepletion of complement C3d, only bands corresponding to the proMBP/AGT and the proMBP/AGT complex were present, suggesting that the additional complexes all involve derivatives of complement C3 (FIG. 4C, lane 2).

### Example 5

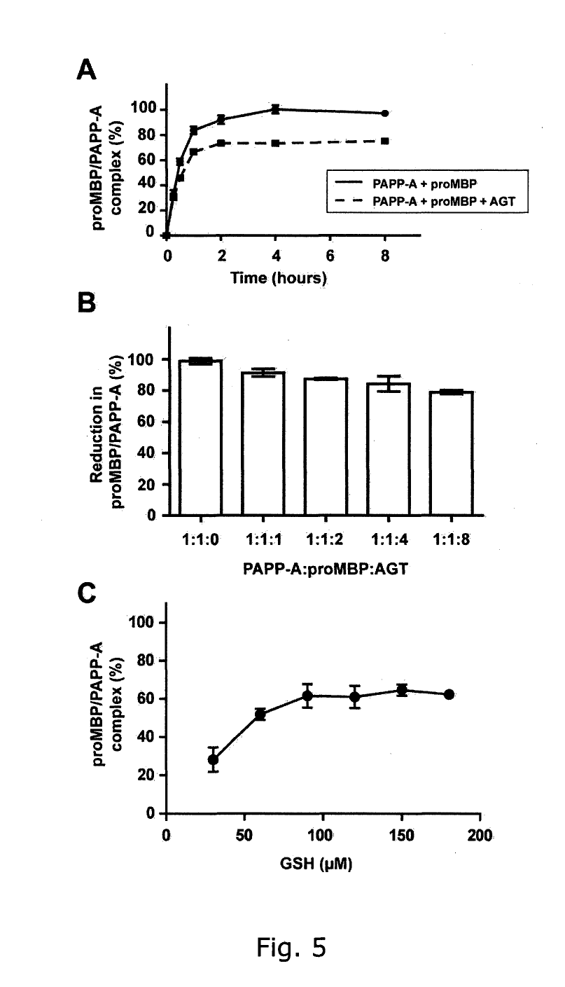

### FIG. 5

Formation of the proMBP/PAPP-A and proMBP/AGT complexes are competing reactions whose balance depends on the redox potential. A, culture supernatants containing recombinant proMBP and PAPP-A were mixed in the absence (molar ratio 1:1:0) or presence (molar ratio 1:1:8) of AGT. The molar concentrations of proMBP and PAPP-A were 20 nM in all experiments. The reactants were incubated at 37° C., and the level of proMBP/PAPP-A complex formed was determined by the complex specific ELISA in samples taken out at defined time points. Reactions were carried out in the presence of 100 μM GSH. B, a similar experiment was carried out varying the molar concentration of AGT, as indicated. Samples were analyzed by the complex specific ELISA following 8 hours of incubation. Concentrations are expressed relative to the amount of complex formed in the absence of AGT, corresponding to the plateau of the experiment in (A). C, a similar experiment, in which the concentration of GSH was varied as indicated. The relative concentration of proMBP/PAPP-A complex formed in the presence of AGT (PAPP-A:proMBP:AGT molar ratio 1:1:12) is plotted as a function of GSH concentration. The data shown are the mean+/− standard deviations of three independent experiments.

In summary, to mimic the conditions of complex formation in vivo, experiments were carried out with all three components. First, recombinant proMBP and PAPP-A were mixed in a 1:1 molar ratio and formation of the proMBP/PAPP-A complex at 37° C. was monitored over time using the proMBP/PAPP-A specific ELISA. Following approximately 2 hours, the concentration of proMBP/PAPP-A complex had reached a maximum (FIG. 5A). Second, a similar experiment was carried out in the presence of an eight-fold molar excess of AGT. In this experiment, the maximum reached was approximately 20% lower (FIG. 5A), showing that AGT is able to compete for proMBP, but that complex formation with PAPP-A is a faster reaction under these conditions. Third, monitoring the amount of proMBP/PAPP-A complex formed after eight hours in the presence of varying concentrations of AGT showed that the inhibitory effect of AGT is dose-dependent (FIG. 5B).

Finally, we asked whether the two reactions of proMBP were affected similarly by changes in the redox potential, as differential sensitivity of the two reactions might determine the balance between the two. An experiment with all three components, similar to the experiment of FIG. 5A, was carried out in the presence of different concentrations of GSH. Indeed, we observed that with decreasing concentration of GSH, the ability of AGT to compete for proMBP is increased (FIG. 5C). Increasing the concentration of GSH to more than 100 μM did not appear to have an effect, but the balance was dramatically affected at concentrations below 100 μM.

### Example 6

Serum and plasma samples collected from pregnant women at various times of gestation was analyzed by complex-specific immunoassays. The concentrations of the following covalent complexes were determined: proMBP/AGT, proMBP/PAPP-A, proMBP/AGT/C3dg. At the time of sampling, no clinical condition was obvious for any of the pregnant women. The concentration of the individual complexes and/or ratios between two or more complexes were calculated and related to the development of clinical conditions developed at a later time of gestation. These conditions included small-for-gestational age, preterm delivery, intrauterine growth restriction IUGR (intrauterine growth restriction), miscarriage, stillbirth, gestational hypertension, HELLP (Haemolysis, Elevated Liver Enzymes and Low Platelet count), preeclampsia, trisomy 13, trisomy 18 and trisomy 21. For assay calibration, purified complexes quantitated by amino acid analyses were used.

### Example 7

### Complexes Containing Proform of Eosinophil Major Basic (proMBP) as Biomarkers of Preeclampsia

### Methods

Blood samples were drawn from 32 pregnant women who developed preeclampsia, defined as de novo hypertension >140/90 mmHg after the 20th week of pregnancy combined with proteinuria >300 mg/L, and a control group of 158 normotensive pregnant women. A maximum of four blood samples were drawn from each woman from the 18^{th} to the 35^{th} week of gestation. Serum was obtained and stored at −80° C. The serum samples were divided into three groups based on the following gestational intervals: <25 weeks, 26-29 weeks, and 30-35 weeks. For each serum sample, the concentrations of the PAPP-A/proMBP complex and the AGT/proMBP complex were measured by specific ELISAs (described above). Dilution series of purified PAPP-A/proMBP complex and the WHO International Reference Preparation 78/610 (Bohn et al., 1980) were used to establish standard curves for the PAPP-A/proMBP specific ELISA and the AGT/proMBP specific ELISA, respectively. In addition to the concentration of the individual complexes, the ratio between the two complexes was calculated for each sample by dividing the concentration of the AGT/proMBP complex with the concentration of the PAPP-A/proMBP: (AGT/proMBP)/(PAPP-A/proMBP). The data was analyzed in GraphPad Prism 5.0.

### Results

All circulating proform of eosinophil major basic protein (proMBP) is known to be covalently bound to either pregnancy-associated plasma protein-A (PAPP-A) or angiotensinogen (AGT) during pregnancy. Here, serum samples were obtained from pregnant women developing preeclampsia and from a control group of normotensive pregnant women. The samples were divided into three gestational intervals: <25 weeks, 26-29 weeks, and 30-35 weeks. To evaluate the diagnostic value of the proMBP complexes as biomarkers of preeclampsia, the concentrations of the PAPP-A/proMBP complex and the AGT/proMBP complex were measured in each serum sample using specific ELISAs. No change in the serum concentrations were observed between the preeclamptic pregnancies and the control group (FIGS. 6A, 7A, and 8A). However, the ratio between the two complexes were significantly different between the two groups (FIGS. 6A, 7A, and 8A). This was observed in all three gestational intervals, but most pronounced from week 30 to week 35 (FIG. 8A).

The specificity and sensitivity of the potential biomarkers were analyzed using receiver operating characteristic (ROC) curves (FIGS. 6B, 7B, and 8B). The area under the curves were calculated. In all three gestational intervals, the ratio between the two measured concentrations—(AGT/proMBP)/(PAPP-A/proMBP)—improved the diagnostic value of the individual complexes, seen by an increased area under the ROC curve. These results suggest that the ratio between the two proMBP complexes could potentially contribute to the prediction of pregnancy complications.

1. A diagnostic and/or prognostic method comprising measurements of the concentrations of two or more different proMBP-complexes in an isolated sample from an individual.

2. The method according to claim 1, wherein the complexes are proMBP/AGT and proMBP/PAPP-A.

3. The method according to claim 1, wherein the complexes are proMBP/AGT and proMBP/AGT/C3dg

4. The method according to claim 1, wherein the complexes are proMBP/PAPP-A and proMBP/AGT/C3dg.

5. The method according to claim 1, wherein the complexes are proMBP/AGT and proMBP/PAPP-A and proMBP/AGT/C3dg.

6.-7. (canceled)

8. The method according to claim 2, wherein a higher concentration and/or a higher ratio of proMBP/AGT compared to the concentration and/or ratio of proMBP/PAPP-A is indicative of disease and/or disease state and/or disease development.

9. The method according to claim 2, wherein a higher concentration and/or ratio of proMBP/PAPP-A compared to the concentration and/or ratio of proMBP/AGT is indicative of disease and/or disease state and/or disease development.

10.-11. (canceled)

12. The method according to claim 1, wherein the concentrations of the individual complexes are indicative for the redox state in an individual.

13. The method according to claim 1, wherein the ratios between the two or more different proMBP-complexes are indicative for the redox state in an individual.

14. The method according to claim 1, wherein a shift in ratio, such as shift in ratio over time, between the two or more different proMBP-complexes is indicative for a change the redox state in an individual.

15. The method according any of claims 12-14 wherein the redox state or the change in redox state is caused by conditions affecting the oxygen tension, such as hypoxia, hyperoxia and hypertension.

16. The method according to claim 1, wherein the disease is a pregnancy associated disease and/or a pregnancy associated complication and/or a genetic abnormality.

17. The method according claim 16, wherein the pregnancy associated disease or complication is selected from the group of diseases/complications consisting of small-for-gestational age, preterm delivery, IUGR (intrauterine growth restriction), miscarriage, stillbirth, gestational hypertension, HELLP (Haemolysis, Elevated Liver Enzymes and Low Platelet count), preeclampsia and any combination thereof.

18. The method according to claim 16, wherein the genetic abnormality is selected from the group of genetic abnormalities consisting of trisomy 13, trisomy 18, trisomy 21 and any combination thereof.

19. (canceled)

20. The method according to claim 19, wherein the individual is a pregnant female.

21. The method according to claim 19, wherein the individual is a fetus.

22. (canceled)

23. The method according to claim 1, wherein the sample is any tissue biopsy, such as a placental biopsy or a biopsy from the umbilical cord.

24. The method according to claim 1, wherein the method comprises the steps of:
a. obtaining an isolated sample from an individual;
b. measuring the concentration and/or ratio of two or more different proMBP-complexes in said isolated sample;
c. obtaining the result; and
d. using the result of step c. to assess and/or determine the disease, the disease state and/or disease development of said individual.

25. The method according to claim 24 wherein the measurements of step b. are performed by ELISA.

26. The method according to claim 24, wherein the measurements of step b. relates to measurement of the PAPP-A activity.

27. The method according to claim 25 or 26, wherein the measurements are combinations of ELISA measurements and PAPP-A activity measurements.

28. A reagent kit comprising detection means for carrying out the method set forth in claim 24, step b.

29. The reagent kit according to claim 28, wherein the detection means are antibodies directed towards proMBP and/or antibodies directed towards AGT and/or antibodies directed towards PAPP-A and/or antibodies directed towards complement C3dg.

30. The reagent kit according to claim 28, wherein the detection means are antibodies directed towards the two or more proMBP-complexes.

31. A method for determination of the redox state of a pregnant female comprising measurement of the concentration of and/or ratio between two or more proMBP-complexes.

32. The method according to claim 31, wherein the proMBP-complexes are the proMBP/AGT complex and the proMBP/PAPP-A complex.

33.-39. (canceled)
